(19) United States
(12) Patent Application Publication
(10) Pub. No.: US 2026/0224401 A1
(43) Pub. Date: Aug. 6, 2026

(54) OPHTHALMIC SURGICAL SYSTEMS AND HANDPIECES

(71) Applicant: Alcon Inc., Fribourg (CH)

(72) Inventors: Edward Troy Tanner, Oceanside, CA (US); Brian William McDonell, Irvine, CA (US)

(21) Appl. No.: 19/447,243

(22) Filed: Jan. 13, 2026

Related U.S. Application Data
(60) Provisional application No. 63/751,357, filed on Jan. 30, 2025.

(51) Int. Cl. A61F 9/007

(52) U.S. Cl. CPC A61F 9/00763

(57) ABSTRACT

In certain embodiments, a system includes a vitrectomy handpiece and a pneumatic assembly coupled to the vitrectomy handpiece. The vitrectomy handpiece includes a cutting assembly, and a diaphragm disposed in a drive chamber and coupled to the cutting assembly. The pneumatic assembly includes an airflow controlling member and an actuating assembly. The airflow controlling member is configured to direct air to the drive chamber to move the diaphragm, thereby actuating the cutting assembly. The actuating assembly is configured to actuate the airflow controlling member.

### INTRODUCTION

Microsurgical procedures frequently require precision cutting and/or removing of various body tissues. For example, certain ophthalmic surgical procedures may require cutting and removing portions of the vitreous humor, a transparent jelly-like material that fills the posterior segment of the eye. The vitreous humor, or vitreous, is composed of numerous microscopic fibrils that are often attached to the retina. Therefore, cutting and removing the vitreous may need to be done with great care to avoid traction on the retina, the separation of the retina from the choroid, a retinal tear, or, in the worst case, cutting and removal of the retina itself. In particular, delicate operations such as mobile tissue management (e.g., cutting and removal of vitreous near a detached portion of the retina or a retinal tear), vitreous base dissection, and cutting and removal of membranes may be particularly difficult.

When removing vitreous, ophthalmic surgeons typically utilize a vitrectomy handpiece to cut the vitreous into smaller fragments, and then suction the fragmented vitreous out of the eye. To cut the vitreous, an inner cutting member of the vitrectomy handpiece is actuated by a pneumatic chamber (and optionally a spring), and moved relative to a port of an outer cutting member. However, due to the design of traditional vitrectomy handpieces, and more particularly, the pneumatic chambers thereof, the pneumatic chamber may limit the cutting capabilities of a vitrectomy handpiece when the operating temperature of the chamber is too high, and/or flow through the chamber is restricted. Such restrictions may reduce the effectiveness of the vitrectomy handpiece.

### BRIEF SUMMARY

The present disclosure relates generally to ophthalmic surgical instruments for performing ophthalmic surgical procedures, such as vitrectomy.

In certain embodiments, a system includes a vitrectomy handpiece and a pneumatic assembly coupled to the vitrectomy handpiece. The vitrectomy handpiece includes a cutting assembly, and a diaphragm disposed in a drive chamber and coupled to the cutting assembly. The pneumatic assembly includes an airflow controlling member and an actuating assembly. The airflow controlling member is configured to direct air to the drive chamber to move the diaphragm, thereby actuating the cutting assembly. The actuating assembly is configured to actuate the airflow controlling member.

In certain embodiments, a vitrectomy handpiece includes a cutting assembly, a diaphragm, and a pneumatic assembly. The diaphragm is disposed in a drive chamber and is coupled to the cutting assembly. The pneumatic assembly is coupled to the cutting assembly and includes an airflow controlling member and an actuating assembly. The airflow controlling member is configured to direct air to the drive chamber to move the diaphragm, thereby actuating the cutting assembly. The actuating assembly is configured to actuate the airflow controlling member.

In certain embodiments, a vitrectomy handpiece includes a handpiece configured to be removably coupled to a cutting assembly. The handpiece includes an actuating assembly coupled to the cutting assembly and configured to actuate the cutting assembly to cut vitreous of an eye. The actuating assembly includes a piezoelectric assembly, and a spring coupled to the piezoelectric assembly and the cutting assembly, where the piezoelectric assembly engages with the spring to cause movement of the cutting assembly.

The following description and the related drawings set forth in detail certain illustrative features of one or more embodiments.

### BRIEF DESCRIPTION OF THE DRAWINGS

The appended figures depict certain aspects of the one or more embodiments and are therefore not to be considered limiting of the scope of this disclosure.

FIG. 1A shows an example of an ophthalmic surgical system used to perform ophthalmic procedures on an eye, according to certain embodiments.

FIG. 1B shows example components of a surgical console of the ophthalmic surgical system of FIG. 1A, according to certain embodiments.

FIG. 2A shows a side view of an example handpiece of the ophthalmic surgical system of FIGS. 1A-1B, according to certain embodiments.

FIG. 2B shows a cross-sectional view of the handpiece of FIG. 2A, according to certain embodiments.

FIGS. 2C-2D illustrate various example distal end configurations of the handpiece shown in FIG. 2B, according to certain embodiments.

FIGS. 3A-3B illustrate cross-sectional views of an example pneumatic assembly coupled to the handpiece of FIG. 2A, according to certain embodiments.

FIG. 4 shows a cross-sectional view of another example pneumatic assembly with a first piezoelectric actuator and a second piezoelectric actuator, and an airflow controlling member for controlling airflow, according to certain embodiments.

FIGS. 5A-5C show cross-sectional views of other example pneumatic assemblies with one or more piezoelectric actuators including protrusions for controlling airflow, according to certain embodiments.

FIG. 6 shows a cross-sectional view of an example pneumatic assembly with a piezoelectric fan for controlling airflow, according to certain embodiments.

FIGS. 7A-7B show cross-sectional views of example pneumatic assemblies with a piezoelectric actuator coupled to an airflow tube for controlling airflow, according to certain embodiments.

FIG. 8A shows a cross-sectional view of an example pneumatic assembly with a wheel for controlling airflow, according to certain embodiments.

FIG. 8B shows a front cross-sectional view of the wheel of FIG. 8A, according to certain embodiments.

FIGS. 9A-9B show cross-sectional views of example handpieces including a piezoelectric assembly and a spring, according to certain embodiments.

To facilitate understanding, identical reference numerals have been used, where possible, to designate identical elements that are common to the drawings. It is contemplated that elements and features of one embodiment may be beneficially incorporated in other embodiments without further recitation.

### DETAILED DESCRIPTION

It will be readily understood that the components of the embodiments as generally described herein and illustrated in the appended Figures can be arranged and designed in a wide variety of different configurations. Thus, the following more detailed description of various embodiments, as represented in the Figures, is not intended to limit the scope of the present disclosure but is merely representative of various embodiments. While the various aspects of the embodiments are presented in the Figures, the Figures are not necessarily drawn to scale unless specifically indicated.

Reference throughout this specification to the term “distal” refers to a system, device, component, end, portion, or segment that is disposed closer to a patient and/or further from a console during an ophthalmic procedure; and the term “proximal” refers to the system, device, component, end, portion, or segment that is disposed further from the patient and/or closer to the console during the ophthalmic procedure.

The vitreous body, often referred to as the vitreous humor or simply “the vitreous,” is a transparent, colorless, and gelatinous mass that fills the space between the lens and the retina of the eyeball (e.g., a posterior segment of the eye). The vitreous makes up about 80% of the volume of the eyeball and helps maintain the round shape of the eye. Additionally, the vitreous assists in absorbing external mechanical shocks to the eye, provides nutrients to the lens, and supports the retina. The vitreous is mostly comprised of water with trace amounts of collagen and hyaluronic acid, which provide the vitreous with its gelatinous structure.

Microsurgical cutting probes used in ophthalmic surgeries that involve removal of the vitreous may include a hollow outer cutting member, a hollow inner cutting member arranged coaxially with and movably disposed within the hollow outer cutting member, a port extending radially through the outer cutting member near the distal end of the outer cutting member, and a port extending radially through the inner cutting member near the distal end of the inner cutting member. Vitreous humor and/or membranes may be aspirated into the open port of the outer cutting member and the inner cutting member may be actuated to extend distally. As the inner cutting member extends distally, cutting surfaces on both the inner and outer cutting members may cooperate to cut the vitreous and/or membranes, and the cut tissue may then be aspirated away through the inner cutting member. Vitreous and/or membranes may then be aspirated into the open ports of both the outer and inner cutting members and the inner cutting member may be actuated to retract proximally. The inner and outer cutting members may cooperate to again cut vitreous and/or membranes and aspirate the cut tissue away.

To actuate the inner cutting member relative to the outer cutting member, conventional vitrectomy handpieces commonly utilize a pneumatic chamber which uses pressurized air to move the inner cutting member in two directions, or a pneumatic chamber which moves in one direction with a spring acting as a restoring force. However, use of the pneumatic chamber to move the inner cutting member may limit the number of actuations, or the cut-rate of the vitrectomy handpiece because the pneumatic chamber can impose a certain amount of resistance to the movement of the inner cutting member.

Accordingly the embodiments described provide vitrectomy handpieces and pneumatic assemblies that provide increased cut-rates at lower operating temperatures, which thereby facilitates quicker, and more efficient removal of vitreous from an eye while also improving the longevity of such handpieces and assemblies.

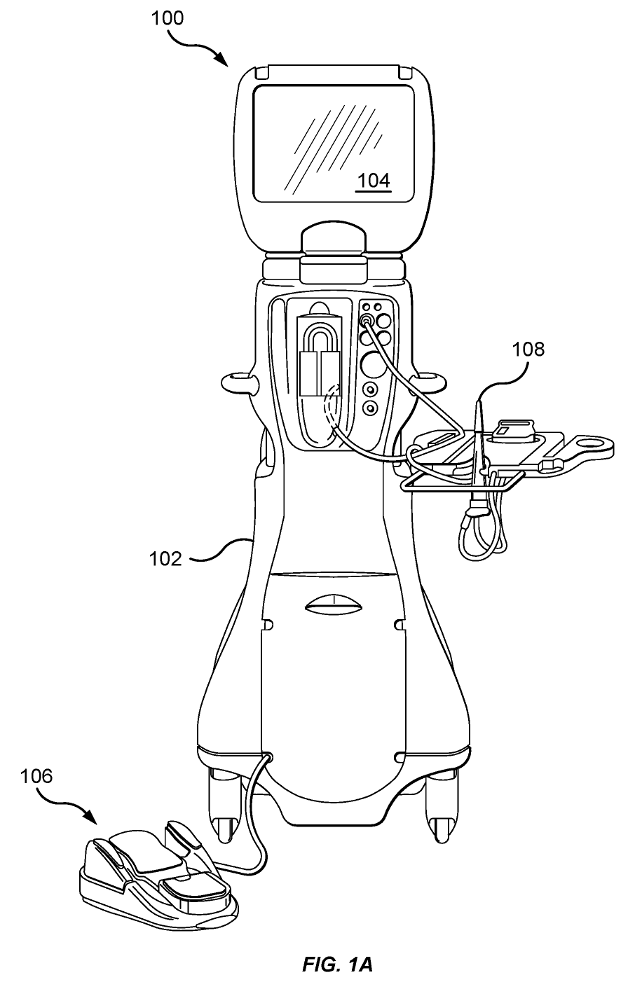

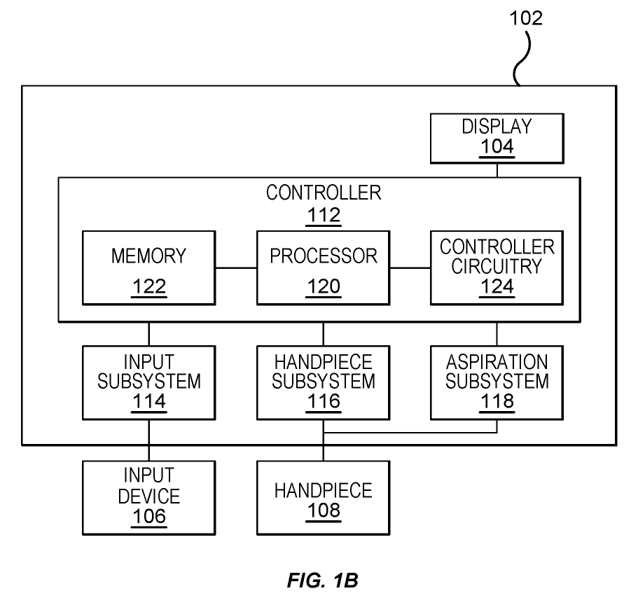

FIG. 1A shows an example ophthalmic surgical system 100 that may be used to perform ophthalmic procedures on an eye, according to certain embodiments. The ophthalmic surgical system 100 includes a console 102 (also referred to as a “surgical console”), which includes a display 104, an input device 106 (e.g., a foot pedal), and a handpiece 108. The components of the ophthalmic surgical system 100 and the surgical console 102 are mechanically and/or electrically coupled as shown and described in more detail with reference to FIG. 1B.

FIG. 1B shows example components of the surgical console 102 of the ophthalmic surgical system 100 shown in FIG. 1A, according to certain embodiments. As shown, the surgical console 102 includes a controller 112, an input subsystem 114, a handpiece subsystem 116, an aspiration subsystem 118, and a display 104. The controller 112 controls the operation of the surgical console 102 and is illustrated as being operationally coupled by a wired or wireless connection to the input device 106 via input subsystem 114, and to the handpiece 108 via handpiece subsystem 116 and aspiration subsystem 118. The controller 112 includes a processor 120, a memory 122, and controller circuitry 124.

The processor 120 may be any type of general purpose processor or could be a processor specifically designed for driving the subsystems illustrated in FIG. 1B, such as an application-specific integrated circuit (“ASIC”). The processor 120 may be, or include, a microprocessor, a microcontroller, an embedded microcontroller, a programmable digital signal processor, or any other programmable device operable to execute instructions stored in the memory 122 for operating surgical console 102. For example, the processor 120 may execute instructions in the memory 122 to receive inputs provided by input device 106 through input subsystem 114 and, in response, send instructions to the aspiration subsystem 118 and/or the handpiece subsystem 116 for operating the handpiece 108. Further, the processor 120 may execute instructions to generate a user interface view for display by the display 104. In some instances, the processor 120 may also be or include a programmable gate array, programmable array logic, or any other device of combinations of devices operable to process electric signals.

The memory 122 can be any type of storage device or non-transitory computer-readable medium, such as random-access memory (“RAM”) or read-only memory (“ROM”), which is operable to receive, store, or recall data, including, but not limited to, electronic, magnetic, or optical memory, whether volatile or non-volatile. The memory 122 stores instructions executed by the processor 120. In example embodiments, functionality disclosed herein can be provided by the processor 120 and the memory 122 (i.e., software based), by the controller circuitry 124 (i.e., hardware based), or by a combination thereof. The memory 122 may include code stored thereon. The code may include instructions that may be executable by the processor 120. The code may be created, for example, using any programming language, including but not limited to, C, C++, Java, Python, Rust, or any other programming language (including assembly languages, hardware description languages, and database programming languages). In some instances, the code may be a program that, when executed by the processor 120, causes the surgical console 102 to operate subsystems 114, 116, and/or 118 for, e.g., driving the handpiece 108 or other devices in communication with the surgical console 102.

The handpiece 108 may be any suitable ophthalmic surgical instrument that can be operated on the basis of the embodiments described herein. For example handpiece 108 may be a vitrectomy handpiece (also referred to as a “vitrectomy probe”).

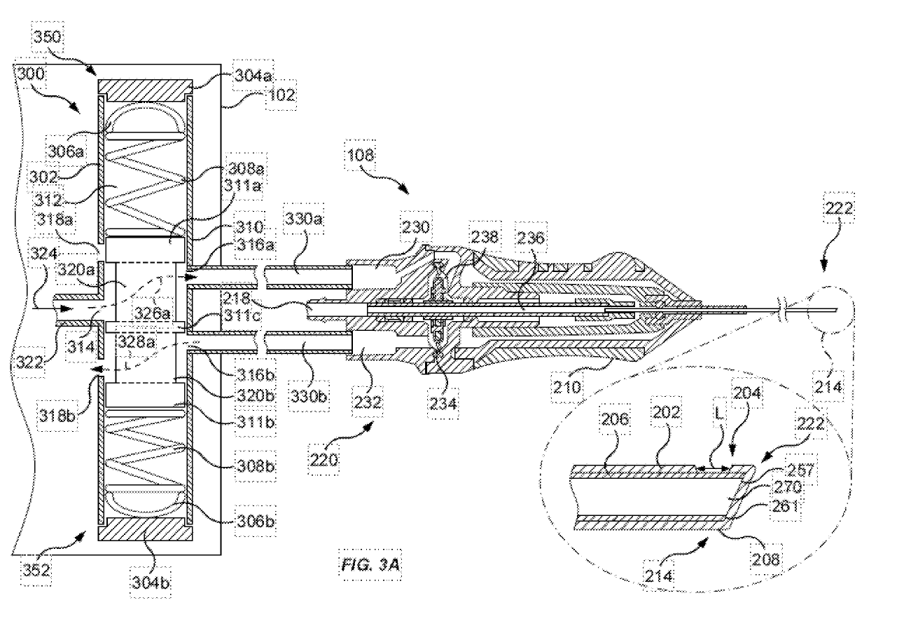

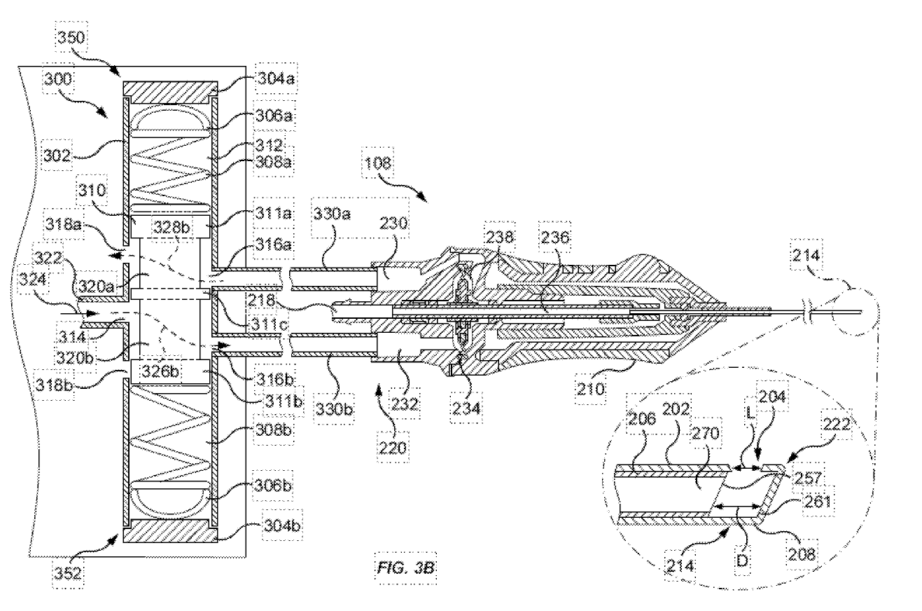

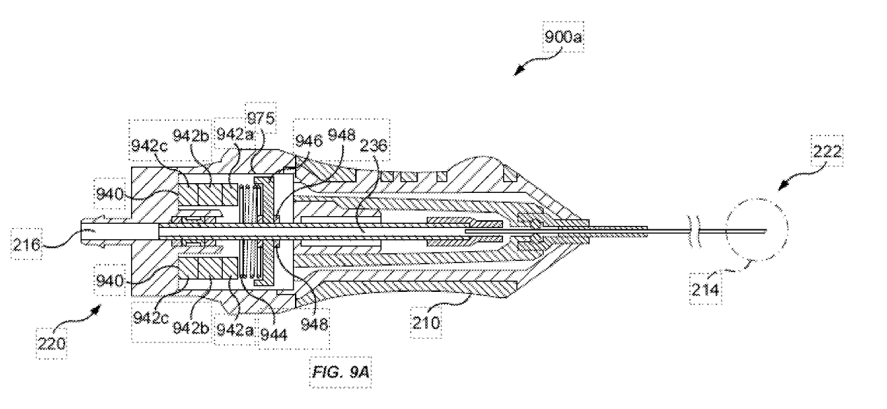

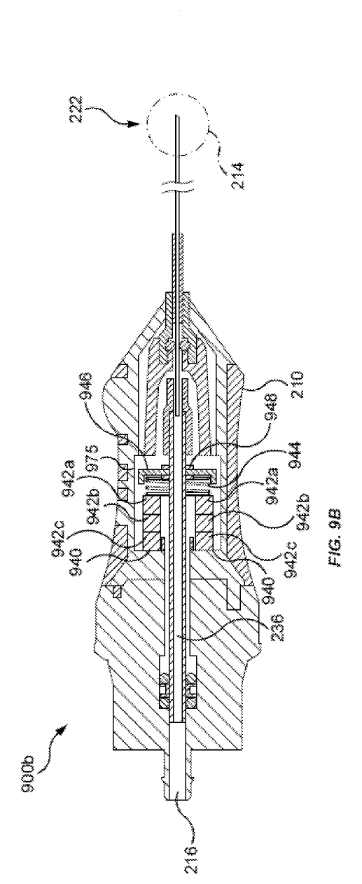

The handpiece subsystem 116 is configured to facilitate the operation of the handpiece 108. In particular, in one example, the handpiece subsystem 116 may control the operations (e.g., oscillatory motion) of a component (e.g., cutting assembly 214) of the handpiece 108. For example, in certain embodiments, the handpiece subsystem 116 communicates with a pneumatic assembly configured to direct air that is delivered to a diaphragm (e.g., diaphragm 234) within the handpiece 108, where the air causes movement of a cutting assembly (e.g., cutting assembly 214) coupled to the diaphragm as described in further detail with reference to FIGS. 3A-3B. In certain other embodiments, the handpiece subsystem 116 applies voltage to a piezoelectric assembly (e.g., piezoelectric assembly 942) to cause movement of the cutting assembly (e.g., cutting assembly 214) coupled to the piezoelectric assembly as described in further detail with reference to FIGS. 9A-9B.

The aspiration subsystem 118 provides aspiration control for the handpiece 108. In some embodiments, the aspiration subsystem 118 may be operatively coupled to a surgical cassette during a surgical procedure. For example, the surgical cassette may be inserted into, attached to, and/or integrated with aspiration subsystem 118 via a coupling mechanism. When aspiration subsystem 118 is operatively coupled to a surgical cassette, aspiration subsystem 118 may control aspiration through the surgical cassette, which may in turn be coupled to the handpiece 108, for example, via a cable or other tether. In certain embodiments, the aspiration subsystem 118 includes one or more mechanical pumps having roller pump heads configured to engage with one or more corresponding pump assemblies on the surgical cassette. The engagement of the roller pump heads and pump assemblies generates a source of pressure and/or vacuum utilized during an ophthalmic surgical procedure.

The input device 106 may be any device that is capable of receiving commands from the user of the surgical console 102 in order to operate the handpiece 108 and/or other components of the surgical console 102. In FIG. 1A, the input device 106 is illustrated as a foot pedal, however, other types of input devices are also within the scope of the disclosure. In one example, the user provides a command to the input device 106, which is received and relayed to the controller 112 by the input subsystem 114. In response, the controller 112 sends instructions to the handpiece subsystem 116 and/or the aspiration subsystem 118 to control the operations of the handpiece 108 based on the user command.

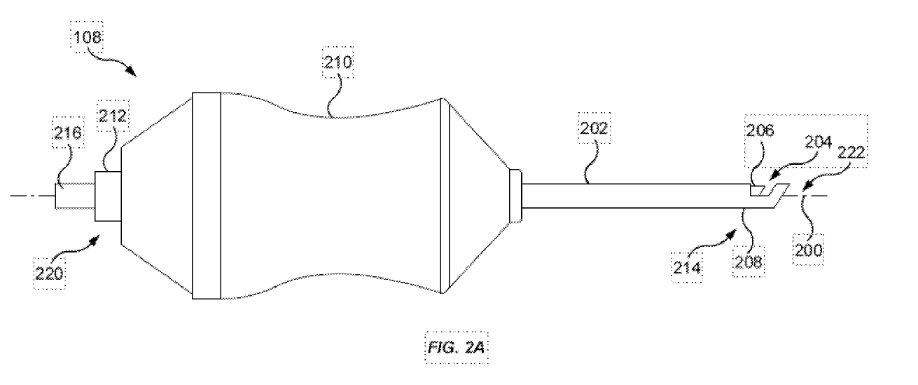

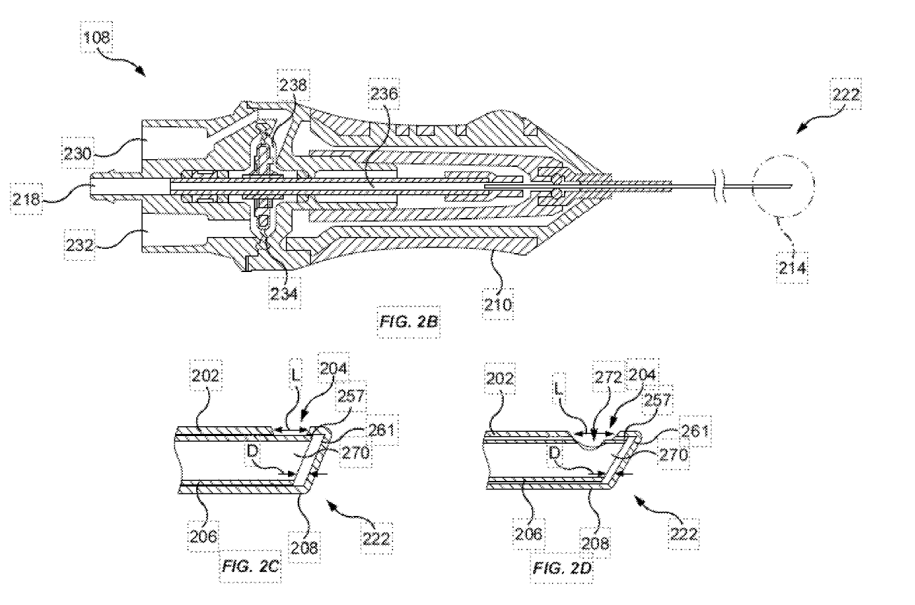

FIG. 2A shows a side view of the handpiece 108 of the ophthalmic surgical system 100 shown in FIGS. 1A-1B, according to certain embodiments. The controller 112 is configured to control the handpiece 108. The handpiece 108 is a vitrectomy handpiece (sometimes referred to herein as a “vitrectomy probe”) which may be used, for example, to cut and/or remove vitreous from inside an eye.

The handpiece 108 includes a cable 216 coupled to the handpiece 108 by a port adapter 212 at a proximal end 220. The cable 216 provides ingress/egress for airflow supply lines and/or vacuum supply lines to be routed into an interior lumen of the handpiece 108. For example, the cable 216 may provide a connection between the handpiece 108, one or more airflow supply lines of an airflow source within the surgical console 102, and a vacuum supply line of a vacuum source within the surgical console 102.

The handpiece 108 further includes a cutting assembly 214, which may be inserted into an eye, e.g., through a trocar cannula, for cutting the vitreous and other tissues and/or materials. The cutting assembly 214 of the handpiece 108 comprises a hollow, outer cutting member 202 (also known as a needle) with a first port 204 at a distal tip 208, and a hollow, inner cutting member 206 disposed within the outer cutting member 202. The outer cutting member 202 and the inner cutting member 206 of the cutting assembly 214 define a major (longitudinal) axis 200 of the handpiece 108.

The first port 204 of the outer cutting member 202 is configured to facilitate the provision of vacuum pressure at a target tissue or material within a patient’s eye for “grabbing” and manipulating the tissue or material during ophthalmic procedures. As shown, the distal tip 208 is beveled (e.g., angled) at an angle that is non-normal relative to the longitudinal axis 200 of the handpiece 108, thereby causing the first port 204 to have an elongated, e.g., ellipsoid shape. The beveled morphology of the distal tip 208 and thus, the elongated shape of the first port 204, increases the surface area of vacuum generation at the first port 204 without requiring an increase in probe gauge. Accordingly, the handpiece 108 enables improved suction or “purchase” of ocular tissues/materials and thus, easier manipulation thereof, at smaller probe gauges. For clarity, enlarged cross-sectional views of the distal tip 208 and the first port 204 are illustrated in FIGS. 2C-2D, which are described in further detail below.

The inner cutting member 206 of the handpiece 108 may be formed of any material suitable for performing ophthalmic procedures. In certain embodiments, the inner cutting member 206 comprises a plastic or polymeric material. In such embodiments, a portion or substantially all of the inner cutting member 206 may be translucent or transparent. In certain other embodiments, the inner cutting member 206 comprises surgical-grade materials, such as aluminum, stainless steel (e.g., 316 or 316L stainless steel), or other alloys. In particular examples, the inner cutting member 206 is formed of Phynox, Elgiloy, or other suitable cobalt-chromium-nickel alloys. In particular examples, the inner cutting member 206 is formed of nitinol or other suitable nickel-titanium alloys. In further embodiments, the inner cutting member 206 may comprise a combination of metallic and polymeric materials.

A proximal end of the inner cutting member 206 may be directly or indirectly attached to a shaft 236 (seen in FIG. 2B) within an interior lumen of housing 210. In certain embodiments, the housing 210 is configured to be held by a user, such as a surgeon. For example, the housing 210 may be ergonomically contoured to substantially fit the hand of the user. In certain embodiments, the outer surface may be textured or have one or more gripping features formed thereon, such as one or more grooves and/or ridges. The housing 210 may be made from any materials commonly used for such instruments and suitable for ophthalmic surgery. For example, the housing 210 may be formed of a lightweight aluminum, a polymer, or other suitable material. In some embodiments, housing 210 may be sterilized and used in more than one surgical procedure, or may be a single-use device.

FIG. 2B shows a cross-sectional view of the handpiece 108 of FIG. 2A, according to certain embodiments. FIGS. 2C-2D illustrate various example distal end configurations of the handpiece 108 shown in FIG. 2B, according to certain embodiments. Accordingly, FIGS. 2B-2D are described together herein for clarity purposes.

The handpiece 108 further includes a first air supply port 230, a second air supply port 232, and an aspiration port 218. The first air supply port 230 and the second air supply port 232 are configured to receive pneumatic pressure (i.e., air flow) to actuate the cutting assembly 214, and the aspiration port 218 provides for aspiration of materials from the cutting assembly 214. Air within the handpiece 108 may also be exhausted via the first air supply port 230 and the second air supply port 232. In certain embodiments, a plurality of tubes disposed within the cable 216 each individually couple the first air supply port 230, the second air supply port 232, and the aspiration port 218 to the console 102.

The handpiece 108 may be a pneumatically driven probe that operates by receiving pneumatic pressure alternating through the first air supply port 230 and the second air supply port 232. The handpiece 108 includes a probe actuator or motor shown here as a reciprocating air driven diaphragm 234 that is coupled to the shaft 236 in an enclosed drive chamber 238. Generally, the inner cutting member 206 may oscillate within the outer cutting member 202 in response to movement of the diaphragm 234 and the shaft 236, which is caused by the alternating pneumatic pressure received through the first air supply port 230 and the second air supply port 232. The alternating pneumatic pressure may be received from a pneumatic assembly, various examples of which are described in further detail with reference to FIGS. 3A-3B, 4, 5A-5C, 6, 7A-7B, and 8A-8B.

Turning to FIGS. 2C-2D, the first port 204 of the outer cutting member 202 is defined by an opening with a length (L) that is between, for example, 1 millimeter (mm) and 10 mm (e.g., between 1.2 mm and 9.8 mm, 1.4 mm and 9.6 mm, 1.6 mm and 9.4 mm, or 1.8 mm and 9.2 mm). The cutting assembly 214 also includes the distal tip 208 with a beveled (i.e., angled) end. The distal tip 208 of the outer cutting member 202 may be closed using, for example, spin closed machining, adhesive, welding (e.g., laser welding), etc. For example, the beveled end may be closed by laser welding a piece onto the angled end. Further, although shown as comprising a beveled end in FIGS. 2C-2D, the distal tip 208 may instead be flat.

As shown in FIGS. 2C-2D, the inner cutting member 206 has a beveled end similar in shape to the distal tip 208 of the outer cutting member 202, and is separated from the distal tip 208 by a distance (D). The distance between the inner cutting member 206 and the outer cutting member 202 changes when the inner cutting member 206 moves relative to an inner angled surface 261 of the outer cutting member 202. Further, the inner cutting member 206 includes a cutting edge 257 and an open end at a distal port 270. The cutting edge 257 is configured to cut any vitreous material which may have been aspirated into the first port 204 of the outer cutting member 202. The distal port 270 is configured to facilitate aspiration of the vitreous material through the inner cutting member 206 and the shaft 236.

As shown in FIG. 2D, the inner cutting member 206 may also include a second port 272 that aligns with the first port 204 of the outer cutting member 202. The second port 272 is configured to facilitate additional cutting and/or aspiration of the vitreous material which enters through the first port 204.

Generally, the inner cutting member 206 may oscillate within the outer cutting member 202 in response to the probe actuator. In an embodiment, the inner cutting member 206 may be driven by air pressure directed on opposing sides of the diaphragm 234. In one example of operation, if air pressure is increased at the first air supply port 230, the diaphragm 234 may move distally, displacing the inner cutting member 206 relative to the outer cutting member 202, thereby moving a cutting edge 257 of the inner cutting member 206 in the distal direction and cutting tissue. Venting the pressure at the first air supply port 230 and increasing the pressure at the second air supply port 232 may move the diaphragm 234 proximally, moving the cutting edge 257 in the proximal direction. Thus, in operation, pneumatic pressure may be directed alternately to the first and second air supply ports 230, 232 to operate the handpiece 108 and actuate the cutting assembly 214.

FIGS. 3A-3B illustrate cross-sectional views of an example pneumatic assembly 300 coupled to the handpiece 108 of FIG. 2A, according to certain embodiments. FIG. 3A illustrates the pneumatic assembly 300 in a first state, where the cutting assembly 214 is closed, and FIG. 3B illustrates the pneumatic assembly 300 in a second state, where the cutting assembly 214 is open. Accordingly, FIGS. 3A-3B are described together herein for clarity.

The pneumatic assembly 300 shown in FIGS. 3A-3B includes a housing 302, a first cap 304a and a second cap 304b (collectively referred to as “caps 304”), a first piezoelectric actuator 306a and a second piezoelectric actuator 306b (collectively referred to as “piezoelectric actuators 306” or “actuating assembly 306”), a first spring 308a and a second spring 308b (collectively referred to as “springs 308”), and an airflow controlling member 310. The caps 304 are at least partially disposed within the housing 302. The piezoelectric actuators 306, springs 308, and the airflow controlling member 310 are disposed within a chamber 312 of the housing 302.

The housing 302 is a cylinder comprised of a non-conductive material (e.g., glass) that functions as an insulator to conductive components of the pneumatic assembly 300. The housing 302 defines the chamber 312 and includes a plurality of slots that define a plurality of airflow paths therethrough based on the state of the pneumatic assembly 300. The housing 302 includes an airflow inlet slot 314, a first tube slot 316a and a second tube slot 316b (collectively referred to herein as “tube slots 316”), and a first exhaust slot 318a and a second exhaust slot 318b (collectively referred to herein as “exhaust slots 318”). The airflow inlet slot 314 is coupled to an airflow inlet port 322, which receives air 324 from an air supply source, e.g., disposed within the console 102. The first tube slot 316a is coupled to a first tube 330a, which is coupled to the first air supply port 230 of the handpiece 108. The second tube slot 316b is coupled to a second tube 330b, which is coupled to the second air supply port 232 of the handpiece 108. In certain embodiments, the exhaust slots 318 are coupled to a muffler where air is exhausted, e.g., to the atmosphere.

The caps 304 are disposed at opposite ends of the housing 302 and at least partially within the housing 302. The first cap 304a is disposed at a first end 350 of the housing 302, and the second cap 304b is disposed at a second end 352 of the housing 302. As an example, the caps 304 are coupled to the housing 302 via an adhesive, ultrasonic welding, or other coupling mechanism. In certain embodiments, the caps 304 are voltage supply caps that are electrically coupled to the piezoelectric actuators 306. Accordingly, the caps 304 are configured to supply a voltage to the corresponding piezoelectric actuators 306.

The piezoelectric actuators 306 are disposed within the housing 302 between the caps 304 and the springs 308. The first piezoelectric actuator 306a is disposed near the first end 350 of the housing 302, and the second piezoelectric actuator 306b is disposed near the second end 352 of the housing 302. As an example, the piezoelectric actuators 306 are comprised of piezoelectric crystals that at least partially deform in response to a voltage applied to the piezoelectric actuators 306 via, e.g., the caps 304.

In certain embodiments, the first piezoelectric actuator 306a and the second piezoelectric actuator 306b are poled oppositely (i.e., have opposite polarization directions), such that the piezoelectric actuators 306 operate out of phase when a voltage is simultaneously applied to the piezoelectric actuators 306. Because of the opposite polarizations, when the piezoelectric actuators 306 are electrically activated, one piezoelectric actuator extends while the other piezoelectric actuator retracts. The piezoelectric actuators 306 extend or retract as a result of the voltage causing the piezoelectric crystals therein to deform (i.e., from their initial, undeformed static state). Which piezoelectric actuator retracts and which piezoelectric actuator extends may be based on, for example, a polarization direction of the piezoelectric actuators 306. Thus, by applying a sinusoidal voltage, the first piezoelectric actuator 306a and the second piezoelectric actuator 306b each alternately transition between an extended state and retracted state (and vice versa). The activation of the piezoelectric actuators 306 is described in further detail below.

The springs 308 are disposed within the housing 302 between the piezoelectric actuators 306 and the airflow controlling member 310. One end of each of the springs 308 engages with one of a corresponding piezoelectric actuator 306, and the other end engages with the airflow controlling member 310. The springs 308 compress and decompress in response to the activation of the piezoelectric actuators 306 and movement of the airflow controlling member 310. In certain embodiments, the springs 308 are compression springs comprised of a conductive material (e.g., beryllium copper). In certain embodiments, the springs 308 are selected according to a desired operating frequency of the pneumatic assembly 300.

The airflow controlling member 310 is disposed within the housing 302 between the springs 308 and the plurality of slots 314, 316, and 318. The airflow controlling member 310 includes a first wall 311a, a second wall 311b, and a third wall 311c (collectively referred to herein as “walls 311”). The first wall 311a and the third wall 311c define a first aperture 320a, and the second wall 311b and the third wall 311c define a second aperture 320b. The first aperture 320a and the second aperture 320b (collectively referred to herein as “apertures 320”) define channels through the airflow controlling member 310 and are configured to align with the plurality of slots 314, 316, and 318 to allow air to flow therethrough based on a state of the pneumatic assembly 300, as described in further detail below.

In certain embodiments, the airflow controlling member 310 is a graphite piston that reduces friction within the housing 302 to nearly zero and increases the Quality (Q) factor of the pneumatic assembly 300, leading to increased displacement of the airflow controlling member 310 (e.g., relative to other non-graphite pistons) when the pneumatic assembly 300 is driven in resonance by the piezoelectric actuators 306.

As illustrated in FIG. 3A, when the pneumatic assembly 300 is in the first state, the first piezoelectric actuator 306a is in an extended state while the second piezoelectric actuator 306b is in a retracted state. For example, voltage is simultaneously applied to the first piezoelectric actuator 306a and the second piezoelectric actuator 306b by the first cap 304a and the second cap 304b, respectively, causing the first piezoelectric actuator 306a to extend and the second piezoelectric actuator 306b to retract. In other words, the first piezoelectric actuator 306a extends longitudinally towards the first spring 308a while the second piezoelectric actuator 306b retracts (or recedes) away from the second spring 308b. Thus, the first piezoelectric actuator 306a engages with the first spring 308a, which then extends to push the airflow controlling member 310 and compress the second spring 308b on the opposing side thereof.

In the first state, the airflow controlling member 310 is positioned such that the first aperture 320a is aligned with the airflow inlet slot 314 and the first tube slot 316a, and the second aperture 320b is aligned with the second tube slot 316b and the second exhaust slot 318b. Accordingly, the airflow controlling member 310 directs the air 324 along an extension airflow path 326a where the air 324 travels from the airflow inlet slot 314, through the first aperture 320a and the first tube slot 316a, and into the first air supply port 230.

When the air 324 travels into the first air supply port 230, the diaphragm 234 moves longitudinally towards the distal end 222 of the handpiece 108. Such movement of the diaphragm 234 results in a similar longitudinal movement of the shaft 236 and the inner cutting member 206. As a result, the inner cutting member 206 extends past the first port 204 of the outer cutting member 202. For example, the cutting edge 257 of the inner cutting member 206 is in complete or almost complete contact with the inner angled surface 261 of the outer cutting member 202, thereby substantially eliminating the gap (shown by distance (D) in FIG. 2C) between the inner cutting member 206 and the outer cutting member 202.

Additionally, in the first state illustrated by FIG. 3A, air within the chamber 238 is exhausted out of the handpiece 108 through the second air supply port 232. In other words, air within the handpiece 108 travels out of the second tube slot 316b and along a first exhaust airflow path 328a, through the second aperture 320b, and out of the second exhaust slot 318b.

As illustrated in FIG. 3B, when the pneumatic assembly 300 is in the second state, the second piezoelectric actuator 306b is in an extended state while the first piezoelectric actuator 306a is in a retracted state. In FIG. 3B, the second piezoelectric actuator 306b is extended and the first piezoelectric actuator 306a is retracted due to the sinusoidal voltage being applied to the oppositely poled piezoelectric actuators 306. Thus, the retraction of the first piezoelectric actuator 306a and the extension of the second piezoelectric actuator 306b thereby causes the second spring 308b to extend and push the airflow controlling member 310, while compressing the first spring 308a on the opposing side thereof.

In the second state, the airflow controlling member 310 is positioned such that the first aperture 320a is aligned with the first tube slot 316a and the first exhaust slot 318a, and the second aperture 320b is aligned with the airflow inlet slot 314 and second tube slot 316b. Accordingly, the airflow controlling member 310 directs the air 324 along a retraction airflow path 326b where the air 324 travels from the airflow inlet slot 314, through the second aperture 320b and the second tube slot 316b, and into the second air supply port 232.

When the air 324 travels into the second air supply port 232, the diaphragm 234 moves longitudinally away (or retracts) from the distal end 222 of the handpiece 108. As a result, the inner cutting member 206 moves back behind the first port 204 of the outer cutting member 202. In other words, the cutting edge 257 is separated from the inner angled surface 261 of the outer cutting member 202 by the distance (D), such that there is a gap between the inner cutting member 206 and the outer cutting member 202.

Additionally, in the second state illustrated by FIG. 3B, air within the chamber 238 is exhausted out of the handpiece 108 through the first air supply port 230. In other words, air within the handpiece 108 travels out of the first tube slot 316a and along a second exhaust airflow path 328b, through the first aperture 320a, and out of the first exhaust slot 318a.

After the pneumatic assembly 300 has reached the second state shown in FIG. 3B, the pneumatic assembly 300 may return to the first state shown in FIG. 3A by continuing to apply the sinusoidal voltage to the piezoelectric actuators 306. The alternating extension and retraction of each of piezoelectric actuators 306 creates a complementary motion that causes oscillating displacement of the airflow controlling member 310. In other words, the airflow controlling member 310 of the pneumatic assembly 300 is configured to be driven in resonance and transition (or move) axially between the positions of the first and second states shown in FIGS. 3A and 3B, respectively, by continuously applying the sinusoidal voltage to the oppositely poled piezoelectric actuators 306. The airflow controlling member 310 may therefore alternate between its two positions very rapidly to alternately provide pneumatic pressure to the first and second air supply ports 230, 232. Such actuation of the pneumatic assembly 300 drives actuation of the cutting assembly 214 by allowing the inner cutting member 206 to achieve a longitudinal oscillatory motion.

In certain embodiments, instead of applying voltage to both the first piezoelectric actuator 306a and the second piezoelectric actuator 306b, the pneumatic assembly 300 alternates between the states shown in FIGS. 3A-3B by alternately applying a voltage to the first piezoelectric actuator 306a and the second piezoelectric actuator 306b. For example, when the pneumatic assembly 300 is in the first state, the first piezoelectric actuator 306a can be activated by the first cap 304a while the second piezoelectric actuator 306b is inactive (i.e., voltage is not applied). And, when the pneumatic assembly 300 is in the second state, the second piezoelectric actuator 306b is activated by the second cap 304b while the first piezoelectric actuator 306a is inactive. When the voltage is no longer applied to one of the piezoelectric actuators 306a or 306b, the piezoelectric crystals return to their initial, undeformed static state.

Once the pneumatic assembly 300 has reached the second state shown in FIG. 3B, the pneumatic assembly 300 may return to the first state shown in FIG. 3A by discontinuing (or stopping) the voltage being applied to the second piezoelectric actuator 306b, and reactivating the voltage applied to the first piezoelectric actuator 306a. Thus, the airflow controlling member 310 of the pneumatic assembly 300 is configured to transition (or move) axially between the positions of the first and second states shown in FIGS. 3A and 3B, respectively, by alternately applying and terminating a voltage provided to the first piezoelectric actuator 306a and the second piezoelectric actuator 306b.

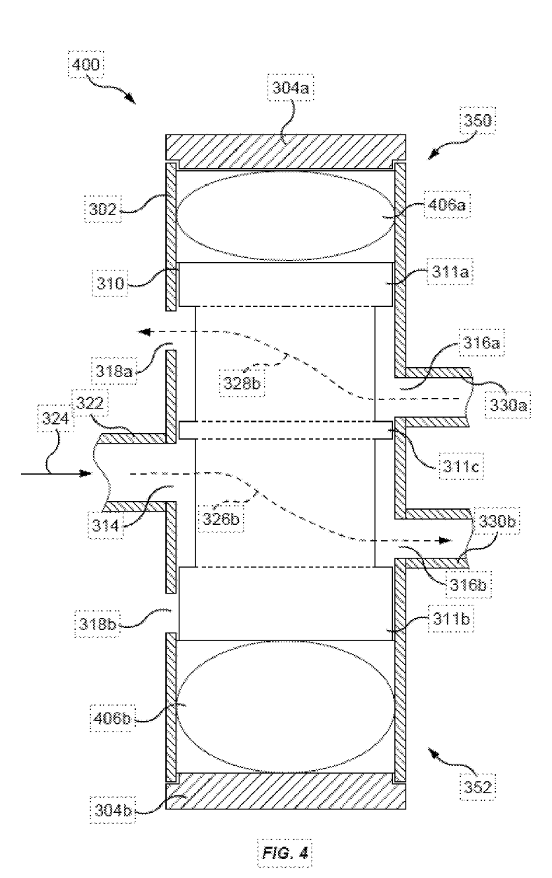

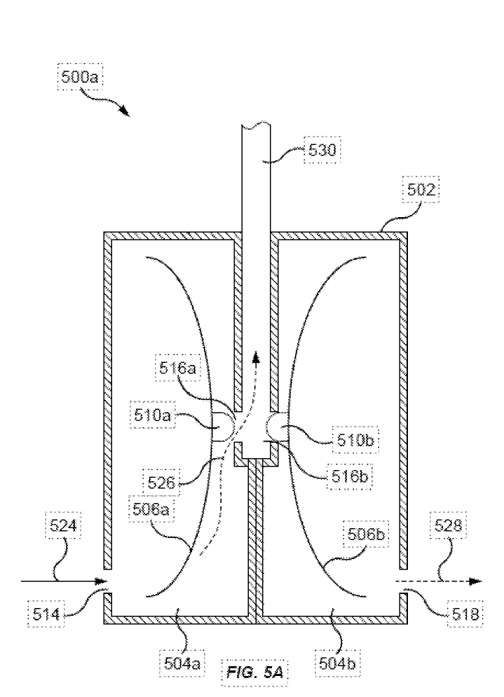

FIG. 4 shows a cross-sectional view of another example pneumatic assembly 400 with a first piezoelectric actuator 406a and a second piezoelectric actuator 406b (collectively referred to herein as “piezoelectric actuators 406” or “actuating assembly 406”), and the airflow controlling member 310 for controlling airflow, according to certain embodiments. The pneumatic assembly 400 may be disposed within the surgical console 102 similar to the pneumatic assembly 300.

The pneumatic assembly 400 shown in FIG. 4 is similar to the pneumatic assembly 300, except the pneumatic assembly 400 does not include the springs 308. Instead, the piezoelectric actuators 406 engage with the airflow controlling member 310 to move the airflow controlling member 310 between the two positions as described with reference to FIGS. 3A-3B. For example, as shown in FIG. 4, the first piezoelectric actuator 406a is in a retracted state and the second piezoelectric actuator 406b is in an extended state, such that the airflow controlling member 310 is in the second state position as described with reference to FIG. 3B. The airflow controlling member 310 can then be moved to the first state position described with reference to FIG. 3A by continuing to apply the sinusoidal voltage to the piezoelectric actuators 406.

In some embodiments, the piezoelectric actuators 406 are configured to drive the airflow controlling member 310 between its two positions very rapidly, or to drive the airflow controlling member 310 in resonance to increase an amplitude of motion of the airflow controlling member 310. As an example, movement of the airflow controlling member 310 can be tuned according a mass of the airflow controlling member 310 and/or a stiffness of the piezoelectric actuators 406.

In certain embodiments, the first piezoelectric actuator 406a and the second piezoelectric actuator 406b may each comprise more than one piezoelectric actuator. For example, the piezoelectric actuators 406 may each comprise a plurality of piezoelectric disks or rings that are stacked face-to-face (similar to the piezoelectric assembly 942 shown in FIGS. 9A-9B).

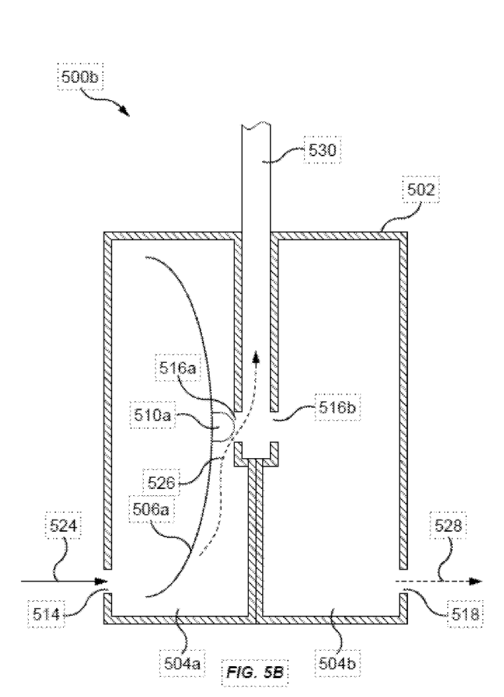

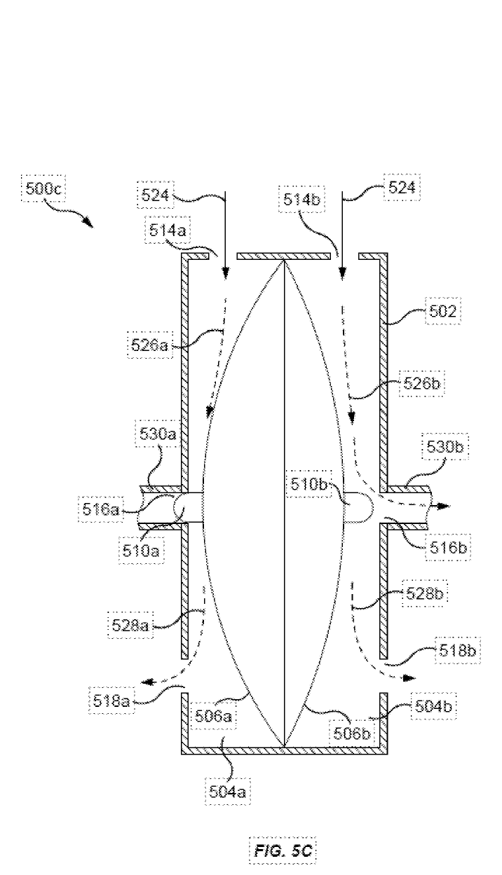

FIGS. 5A-5C show cross-sectional views of other example pneumatic assemblies 500a, 500b, and 500c (collectively referred to herein as “pneumatic assemblies 500”) with one or more piezoelectric actuators 506 including protrusions 510 for controlling airflow, according to certain embodiments.

For clarity, only one set of components (or half) of the pneumatic assemblies 500a, 500b are shown in FIGS. 5A-5B, but the pneumatic assemblies 500a, 500b include an additional set of the same or similar components which operate similar thereto as described herein. In other words, a pneumatic assembly is associated with each of the two air supply ports 230, 232. For example, each of the pneumatic assemblies 500a, 500b includes an additional set of components so that one tube is coupled to the first air supply port 230, and another tube is coupled to the second air supply port 232 (similar to the pneumatic assemblies 300, 400). These embodiments may operate similar to the manner described above with respect to FIGS. 3A-3B, with the assemblies being configured to simultaneously and/or independently receive operating signals from the controller 112.

As illustrated in FIG. 5A, the pneumatic assembly 500a includes a housing 502 having a first chamber 504a and a second chamber 504b (collectively referred to herein as “chambers 504”). The first chamber 504a defines an airflow inlet slot 514 and a first tube slot 516a, and the second chamber 504b defines a second tube slot 516b and an exhaust slot 518. Air 524 enters the first chamber 504a through the airflow inlet slot 514, and exits through the exhaust slot 518. In certain embodiments, the airflow inlet slot 514 is coupled to an air supply source, e.g., within the console 102, and the exhaust slot 518 is coupled to a muffler. A tube 530 is disposed between the chambers 504 and extends from the housing 502. In certain embodiments, the tube 530 connects to the first air supply port 230 or the second air supply port 232 of the handpiece 108 shown in FIG. 2B.

A first piezoelectric actuator 506a is mounted in the first chamber 504a and a second piezoelectric actuator 506b is mounted in the second chamber 504b. The first piezoelectric actuator 506a and the second piezoelectric actuator 506b (collectively referred to herein as “piezoelectric actuators 506”) include a first protrusion 510a and a second protrusion 510b (collectively referred to herein as “protrusions 510”) attached respectively thereto. The protrusions 510 may be comprised of an elastomeric material or other similar rubber material, and may be attached to the piezoelectric actuators 506, for example, via an adhesive, ultrasonic welding, or other coupling mechanism. In certain embodiments, the protrusions 510 are inserted through a hole that is drilled through each of the piezoelectric actuators 506, such that a tail feature of the protrusions 510 secure the protrusions 510 to the piezoelectric actuators 506.

The protrusions 510 extend laterally from the piezoelectric actuators 506 and are aligned with the tube slots 516. As such, the protrusions 510 are configured to plug (or fill) the tube slots 516 in response to activation of the piezoelectric actuators 506. Thus, the protrusions 510 may collectively function as an airflow controlling member because the protrusions 510 control whether the air 524 enters or exits the tube 530 coupled to the handpiece 108.

During operation of the pneumatic assembly 500a, the piezoelectric actuators 506 alternately move the first protrusion 510a into the first tube slot 516a and the second protrusion 510b into the second tube slot 516b. For example, when the first piezoelectric actuator 506a is in a retracted state and the second piezoelectric actuator 506b is in an extended state as shown in FIG. 5A, the air 524 travels along an airflow path 526 because the first tube slot 516a is not blocked by the first protrusion 510a and the second tube slot 516b is blocked (or plugged) by the second protrusion 510b. The airflow path 526 may be an extension airflow path or a retraction airflow path based on whether the tube 530 is coupled to the first air supply port 230 or the second air supply port 232. In other words, when the tube 530 is coupled to the first air supply port 230, the airflow path 526 is an extension airflow path, and when the tube 530 is coupled to the second air supply port 232, the airflow path 526 is a retraction airflow path.

In certain embodiments, when the first piezoelectric actuator 506a transitions to the extended state, its piezoelectric crystals deform to move the first protrusion 510a into the first tube slot 516a, and the second piezoelectric actuator 506b transitions to the retracted state to pull the second protrusion 510b away from (or out of) the second tube slot 516b. As such, air within the handpiece 108 can exit through the pneumatic assembly 500a along an exhaust airflow path 528 and out of the exhaust slot 518.

Turning to FIG. 5B, the pneumatic assembly 500b is similar to the pneumatic assembly 500a, except the pneumatic assembly 500b does not include the second piezoelectric actuator 506b and the second protrusion 510b (seen in FIG. 5A). Instead, the pneumatic assembly 500a controls the air 524 directed therethrough using the first piezoelectric actuator 506a and the first protrusion 510a. For example, the first piezoelectric actuator 506a may be activated so that the first protrusion 510a plugs the first tube slot 516a as described with reference to FIG. 5A.

In certain embodiments, the pneumatic assemblies 500a, 500b are operated in phase with the additional components so that the air 524 is alternately directed along a retraction airflow path and an extension airflow path. Such operation of the pneumatic assemblies 500a, 500b drives actuation of the cutting assembly 214 by alternately directing the air 524 into the air supply ports 230, 232, which causes the inner cutting member 206 to achieve a longitudinal oscillatory motion.

In certain embodiments, the pneumatic assemblies 500a, 500b do not include another set of components associated with an air supply port. For example, the pneumatic assemblies 500a, 500b may be coupled to the first air supply port 230 to direct the air 524 along an extension airflow path and extend the inner cutting member 206 distally. To return (or retract) the inner cutting member 206, the handpiece 108 may include a spring disposed therein. Accordingly, the spring may be configured to engage with the shaft 236 to cause the inner cutting member 206 to retract proximally after actuation by the pneumatic assemblies 500a, 500b.

Turning to FIG. 5C, the pneumatic assembly 500c is similar to the pneumatic assembly 500a, except the piezoelectric actuators 506 of the pneumatic assembly 500c are disposed back-to-back within the housing 502, where the first piezoelectric actuator 506a defines the first chamber 504a and the second piezoelectric actuator 506b defines the second chamber 504b. The pneumatic assembly 500c also includes a first airflow inlet slot 514a and a second airflow inlet slot 514b (collectively referred to herein as “airflow inlet slots 514”), a first tube slot 516a and a second tube slot 516b (collectively referred to herein as “tube slots 516”), as well as a first exhaust slot 518a and a second exhaust slot 518b (collectively referred to herein as “exhaust slots 518”), and a first tube 530a and a second tube 530b (collectively referred to herein as “tubes 530”).

The first chamber 504a defines the first airflow inlet slot 514a and the first exhaust slot 518a. The second chamber 504b defines the second airflow inlet slot 514b and the second exhaust slot 518b. The first tube 530a is coupled to the first tube slot 516a and the second tube 530b is coupled to the second tube slot 516b. In certain embodiments, the first tube 530a and the second tube 530b are respectively coupled to the first air supply port 230 and the second air supply port 232 of the handpiece 108 (similar to the tubes 330 in FIGS. 3A-3B).

During operation of the pneumatic assembly 500c, the piezoelectric actuators 506 are alternately activated to move the first protrusion 510a into the first tube slot 516a and the second protrusion 510b into the second tube slot 516b. For example, when the first piezoelectric actuator 506a is in an extended state and the second piezoelectric actuator 506b is in a retracted state as shown in FIG. 5C, the air 524 travels along a retraction airflow path 526b because the second tube slot 516b is not blocked by the second protrusion 510b and the first tube slot 516a is blocked (or plugged) by the first protrusion 510a. When the first tube slot 516a is blocked, the air 524 in the first chamber 504a travels along a first exhaust airflow path 528a through the first exhaust slot 518a.

In certain embodiments, when the second piezoelectric actuator 506b transitions to the extended state, its piezoelectric crystals deform to move the second protrusion 510b into the second tube slot 516b, and the first piezoelectric actuator 506a transitions to the retracted state to pull the first protrusion 510a away from (or out of) the first tube slot 516a. As such, the air 524 can travel through the first chamber 504a along an extension airflow path 526a through the first tube slot 516a, and out of the second chamber 504b along the second exhaust airflow path 528b through the second exhaust slot 518b.

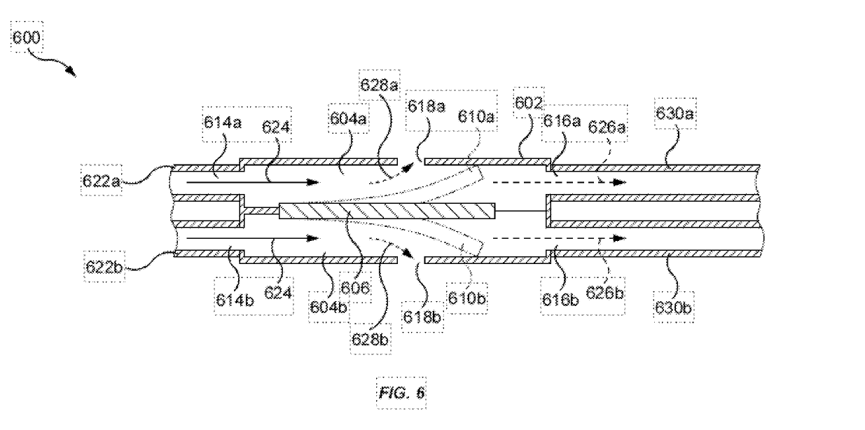

FIG. 6 shows a cross-sectional view of another example pneumatic assembly 600 with a piezoelectric actuator 606 for controlling airflow, according to certain embodiments.

The pneumatic assembly 600 shown in FIG. 6 includes a housing 602 having a first chamber 604a and a second chamber 604b (collectively referred to herein as “chambers 604”) with the piezoelectric actuator 606 mounted therebetween. The pneumatic assembly 600 also includes a first airflow inlet slot 614a and a second airflow inlet slot 614b (collectively referred to herein as “airflow inlet slots 614”), a first tube slot 616a and a second tube slot 616b (collectively referred to herein as “tube slots 616”), as well as a first exhaust slot 618a and a second exhaust slot 618b (collectively referred to herein as “exhaust slots 618”), a first airflow inlet port 622a and a second airflow inlet port 622b (collectively referred to herein as “airflow inlet ports 622”), and a first tube 630a and a second tube 630b (collectively referred to herein as “tubes 630”). In certain embodiments, the first tube 630a and the second tube 630b are respectively coupled to the first air supply port 230 and the second air supply port 232 of the handpiece 108 (similar to the tubes 330 in FIGS. 3A-3B).

Air 624 enters the housing 602 via the airflow inlet slots 614 and may flow through the tube slots 616 and the tubes 630 based on a position of the piezoelectric actuator 606. The piezoelectric actuator 606 is a piezoelectric fan that functions as the airflow controlling member. For example, the piezoelectric actuator 606 is configured to transition between a first position 610a and a second position 610b. In other words, actuation of the piezoelectric actuator 606 causes the piezoelectric actuator 606 to alternately move between the first position 610a and the second position 610b, thereby alternately blocking the air 624 from entering the first tube 630a and the second tube 630b. In certain embodiments, the piezoelectric actuator 606 is driven in resonance and can be operated at various predetermined frequencies.

When the pneumatic assembly 600 is in a first state, the piezoelectric actuator 606 is in the first position 610a. As such, in the first state, the air 624 flowing into the first chamber 604a travels along a first exhaust airflow path 628a and out through the first exhaust slot 618a, and the air 624 flowing into the second chamber 604b travels along a retraction airflow path 626b through the second tube slot 616b. When the pneumatic assembly 600 is in a second state, the piezoelectric actuator 606 is in the second position 610b. As such, in the second state, the air 624 flowing into the second chamber 604b travels along a second exhaust airflow path 628b and out through the second exhaust slot 618b, and the air 624 flowing into the first chamber 604a travels along an extension airflow path 626a through the first tube slot 616a.

Thus, the piezoelectric actuator 606 is configured to transition (or move) between the first state and second state shown by the first position 610a and the second position 610b, respectively, by continuously applying a sinusoidal voltage to the piezoelectric actuator 606. Such actuation of the pneumatic assembly 600 drives actuation of the cutting assembly 214 by alternately directing the air 624 into the air supply ports 230, 232, which causes the inner cutting member 206 to achieve a longitudinal oscillatory motion.

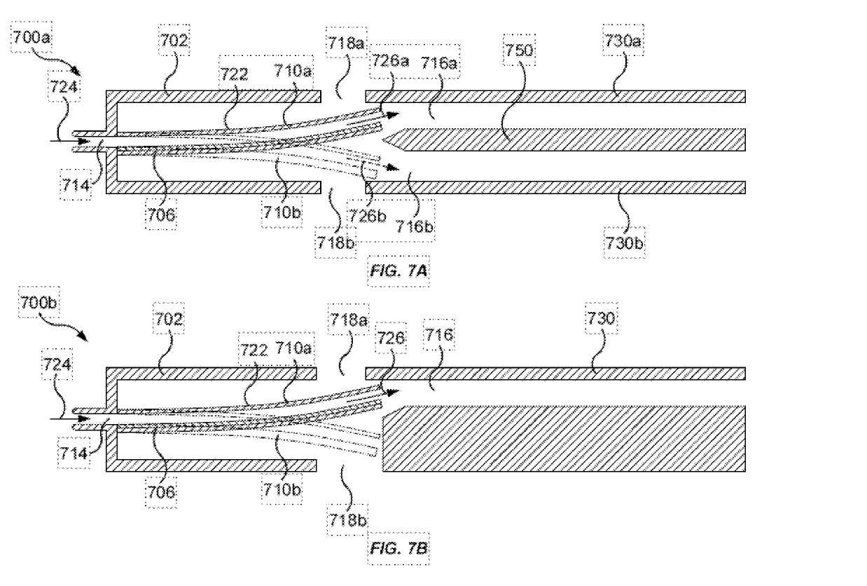

FIGS. 7A-7B show cross-sectional views of other example pneumatic assemblies 700a, 700b with a piezoelectric actuator 706 coupled to an airflow tube 722 for controlling airflow, according to certain embodiments.

The pneumatic assembly 700a shown in FIG. 7A includes a housing 702 with the piezoelectric actuator 706 mounted therein. The pneumatic assembly 700 also includes an airflow inlet slot 714, a first tube slot 716a and a second tube slot 716b (collectively referred to herein as “tube slots 716”), as well as a first exhaust slot 718a and a second exhaust slot 718b (collectively referred to herein as “exhaust slots 718”), and a first tube 730a and a second tube 730b (collectively referred to herein as “tubes 730”) separated by a divider 750. In certain embodiments, the first tube 730a and the second tube 730b are respectively coupled to the first air supply port 230 and the second air supply port 232 of the handpiece 108 (similar to the tubes 330 in FIGS. 3A-3B).

The airflow tube 722 is coupled to the piezoelectric actuator 706, e.g., via an adhesive, ultrasonic welding, or other coupling mechanism. The airflow tube 722 is comprised of a flexible material that is configured to move (or bend) in response to movement of the piezoelectric actuator 706. The airflow tube 722 is coupled to the airflow inlet slot 714, such that air 724 enters the airflow tube 722 via the airflow inlet slot 714 and may be output into one of the tubes 730 based on a position of the piezoelectric actuator 706. As such, in the pneumatic assembly 700a, the airflow tube 722 functions as the airflow controlling member.

The piezoelectric actuator 706 is a piezoelectric fan that functions similar to the piezoelectric actuator 606 of FIG. 6. Thus, actuation of the piezoelectric actuator 706 causes the piezoelectric actuator 706 and the airflow tube 722 to alternately move between a first position 710a and a second position 710b, thereby alternately directing the air 724 into the first tube 730a and the second tube 730b.

When the pneumatic assembly 700 is in a first state, the piezoelectric actuator 706 and the airflow tube 722 are in the first position 710a. As such, in the first state, the air 724 output by the airflow tube 722 travels along an extension airflow path 726a through the first tube slot 716a, and air within the handpiece 108 exits the pneumatic assembly 700 through the second exhaust slot 718b. When the pneumatic assembly 700 is in a second state, the piezoelectric actuator 706 and the airflow tube 722 are in the second position 710b. As such, in the second state, the air 724 output by the airflow tube 722 travels along a retraction airflow path 726b through the second tube slot 716b, and air within the handpiece 108 exits the pneumatic assembly 700 through the first exhaust slot 718a.

Thus, the piezoelectric actuator 706 of the pneumatic assembly 700a is configured to transition (or move) between the positions 710a, 710b of the first and second states by continuously applying a sinusoidal voltage to the piezoelectric actuator 706. Such actuation of the pneumatic assembly 700a drives actuation of the cutting assembly 214 by alternately directing the air 724 into the air supply ports 230, 232, which causes the inner cutting member 206 to achieve a longitudinal oscillatory motion.

Turning to FIG. 7B, for clarity, only one set of components (or half) of the pneumatic assembly 700b is shown in FIG. 7B, but the pneumatic assembly 700b includes an additional set of the same or similar components which operate similar thereto as described herein. In other words, a pneumatic assembly is associated with each of the two air supply ports 230, 232. For example, the pneumatic assembly 700b includes an additional set of components so that one tube is coupled to the first air supply port 230, and the other tube is coupled to the second air supply port 232 (similar to the pneumatic assemblies 500a, 500b). These embodiments may operate similar to the manner described, with the assemblies being configured to independently receive operating signals from the controller 112.

The pneumatic assembly 700b shown in FIG. 7B is similar to the pneumatic assembly 700a, except the pneumatic assembly 700b directs the air 724 into the tube 730 or out of the second exhaust slot 718b. Thus, the pneumatic assembly 700b controls output of the air 724 by alternately directing the air 724 along an airflow path 726 through the tube 730, or out of the housing 702 through the second exhaust slot 718b. The airflow path 726 may be an extension airflow path or a retraction airflow path based on whether the tube 730 is coupled to the first air supply port 230 or the second air supply port 232. In other words, when the tube 730 is coupled to the first air supply port 230, the airflow path 726 is an extension airflow path, and when the tube 730 is coupled to the second air supply port 232, the airflow path 726 is a retraction airflow path.

In certain embodiments, the pneumatic assembly 700b is operated in phase so that the air 724 is alternately directed along a retraction airflow path and an extension airflow path. Such operation of the pneumatic assembly 700b drives actuation of the cutting assembly 214 by alternately directing the air 724 into the air supply ports 230, 232, which causes the inner cutting member 206 to achieve a longitudinal oscillatory motion.

In certain embodiments, the piezoelectric actuator 706 extends partially along a length of the airflow tube 722, such that an excitation signal can be applied thereto to oscillate the airflow tube 722 in resonance (e.g., between the positions 710). In certain embodiments, the pneumatic assembly 700b does not include another set of components associated with an air supply port. For example, the pneumatic assembly 700b may be coupled to the first air supply port 230 to direct the air 724 along an extension airflow path and extend the inner cutting member 206 distally. To return (or retract) the inner cutting member 206, the handpiece 108 may include a spring disposed therein. Accordingly, the spring may be configured to engage with the shaft 236 to cause the inner cutting member 206 to retract proximally after actuation by the pneumatic assembly 700b.

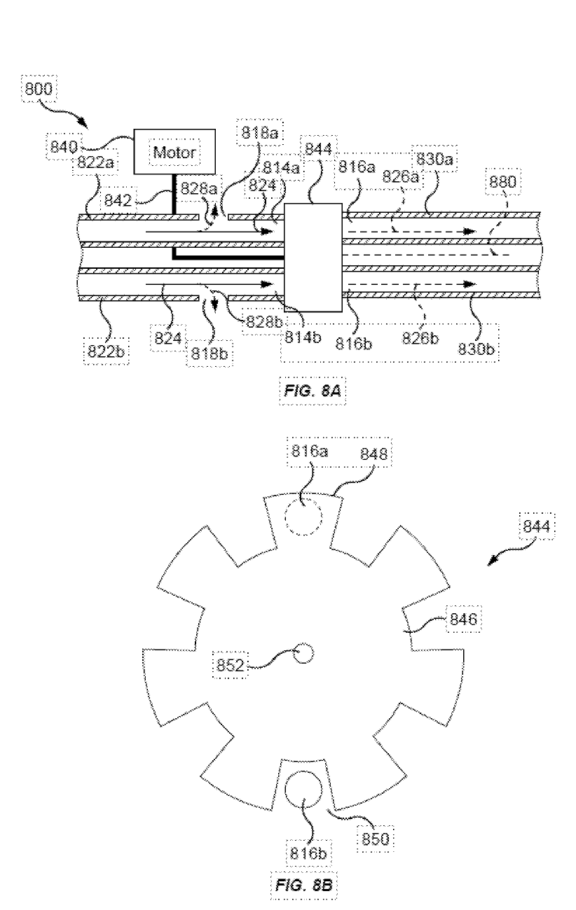

FIG. 8A shows a cross-sectional view of an example pneumatic assembly 800 with a wheel 844 for controlling airflow, according to certain embodiments. FIG. 8B shows a front cross-sectional view of the wheel 844 of FIG. 8A, according to certain embodiments.

The pneumatic assembly 800 shown in FIG. 8A includes a motor 840 operably coupled to the wheel 844 via a cable 842. The pneumatic assembly 800 also includes a first airflow tube 822a and a second airflow tube 822b (collectively referred to herein as “airflow tubes 822”) aligned with a first tube 830a and a second tube 830b (collectively referred to herein as “tubes 830”). The first airflow tube 822a includes a first airflow inlet slot 814a and a first exhaust slot 818a. The second airflow tube 822b includes a second airflow inlet slot 814b and a second exhaust slot 818b. Further, the first tube 830a includes a first tube slot 816a, and the second tube 830b includes a second tube slot 816b. In certain embodiments, the first tube 830a and the second tube 830b are respectively coupled to the first air supply port 230 and the second air supply port 232 of the handpiece 108 (similar to the tubes 330 in FIGS. 3A-3B).

The wheel 844 is disposed between the airflow tubes 822 and the tubes 830, and is configured to rotate about a longitudinal axis 880. As shown in FIG. 8B, the wheel includes a body 846, a plurality of protrusions 848, and a plurality of indentations 850.

The body 846 is coupled to a center rod 852, which is coupled to the cable 842 and configured to rotate the wheel 844 in response to actuation by the motor 840. In certain embodiments, the motor 840 is operably coupled to the controller 112 of the surgical console 102. The protrusions 848 and the indentations 850 are alternately disposed circumferentially around the wheel 844 and are configured to align with the first airflow inlet slot 814a and the second airflow inlet slot 814b based on a position of the wheel 844 during rotation. While the protrusions 848 protrude laterally outwards from the body 846 and block the air 824 from traveling along an extension airflow path 826a and a retraction airflow path 826b, the indentations 850 allow the air 824 to travel along the extension airflow path 826a and the retraction airflow path 826b. Accordingly, the air 824 flowing through the airflow tubes 822 is either: (a) blocked at the first airflow inlet slot 814a and allowed to flow through the second airflow inlet slot 814b as shown in FIG. 8B; or (b) allowed to flow through the first airflow inlet slot 814a and blocked at the second airflow inlet slot 814b.

When the air 824 is blocked at the first airflow inlet slot 814a and is allowed to flow through the second airflow inlet slot 814b as shown in FIG. 8B, the air 824 travels along a first exhaust airflow path 828a out the first exhaust slot 818a of the first airflow tube 822a, and along the retraction airflow path 826b through the second tube 830b because the protrusion 848 blocks the first tube slot 816a and the indentation 850 permits the air 824 to flow through the second tube slot 816b. Alternatively, when the air 824 is allowed to flow through the first airflow inlet slot 814a and is blocked at the second airflow inlet slot 814b, the air 824 travels along the extension airflow path 826a through the first tube 830a, and along a second exhaust airflow path 828b out the second exhaust slot 818b of the second airflow tube 822b because the indentation 850 permits the air 824 to flow through the first tube slot 816a and the protrusion 848 blocks the second tube slot 816b.

In certain embodiments, the pneumatic assembly 800 is operated so that the rotation of the wheel 844 causes the air 824 to be alternately directed along the extension airflow path 826a and the retraction airflow path 826b. Such operation of the pneumatic assembly 800 drives actuation of the cutting assembly 214 by alternately directing the air 824 into the air supply ports 230, 232, which causes the inner cutting member 206 to achieve a longitudinal oscillatory motion. Because the wheel 844 can be rotated at high speeds (e.g., greater than 2,800 rotations per minute (RPM)), the air 824 driving the cutting assembly 214 can be alternately delivered at high frequencies. In certain embodiments the spacing and/or sizing of the protrusions 848 and indentations 850 can be adjusted to control the delivery of the air 824 to the first and second air supply ports 230, 232.

In certain embodiments, the pneumatic assemblies 500, 600, 700, and 800 shown in FIGS. 5, 6, 7A-7B, and 8 may be disposed within the surgical console 102 or within the handpiece 108. When the pneumatic assemblies 500, 600, 700, and 800 are disposed within the surgical console 102, the pneumatic assemblies 500, 600, 700, and 800 are coupled to the air supply source within the surgical console 102, and are coupled to the first and second air supply ports 230, 232 of the handpiece 108 via the cable 216. When the pneumatic assemblies 500, 600, 700, and 800 are disposed within the handpiece 108, the pneumatic assemblies 500, 600, 700, and 800 are coupled to the air supply source within the surgical console 102 via the cable 216, and are coupled to the first and second air supply ports 230, 232 within the handpiece 108.

In certain embodiments, use of the pneumatic assemblies 300, 400, 500, 600, 700, and 800 to actuate the cutting assembly 214, as described herein, enables the handpiece 108 to achieve greater cut-rates while also maintaining a stable operating temperature of the pneumatic assemblies. As a result of increased cut-rates provided by the pneumatic assemblies 300, 400, 500, 600, 700, and 800, the handpiece 108 experiences improved efficiency when removing vitreous from the eye. Further, by operating at stable operating temperatures, the pneumatic assemblies 300, 400, 500, 600, 700, and 800 experience improved longevity and reliability as the components therein are less likely to overheat and/or need to be replaced.

FIGS. 9A-9B show cross-sectional views of example handpieces 900a, 900b (collectively referred to herein as “handpieces 900”) including a plurality of piezoelectric actuators 942a, 942b, 942c (942a-c) (collectively referred to herein as piezoelectric assembly 942) and a spring 944, according to certain embodiments.

The handpiece 900a shown in FIG. 9A is similar to the handpiece 108 shown in FIG. 2B, but is driven by the piezoelectric assembly 942 and the spring 944, which are disposed in an enclosed drive chamber 975. The piezoelectric actuators 942a-c comprise piezoelectric disks or rings that are stacked face-to-face and may be bonded together via, for example, epoxy or glass beads. Each of the piezoelectric actuators 942a-c may be separately electrically coupled to the controller 112. Within the enclosed drive chamber 975, a proximal end of the piezoelectric assembly 942 abuts, and is attached (e.g., via an adhesive, ultrasonic welding, or other coupling mechanism) to a hard stop surface 940 of the housing 210, and a distal end of the piezoelectric assembly 942 abuts, and is attached to the spring 944. The spring 944 is attached to a moveable anchor 946. The anchor 946 is disposed over the distal end of the spring 944 and within a set of shaft grooves 948 laterally protruding from the shaft 236, which is coupled to the inner cutting member 206 of the cutting assembly 214 as described above with reference to FIG. 2B.

Generally, the cutting assembly 214 is actuated in response to movement of the shaft 236, which is caused by activation of one or more of the piezoelectric actuators 942a-c. As illustrated in FIG. 3A above, when the cutting assembly 214 is in the first state, the cutting assembly 214 is closed. In the closed, first state a voltage is being applied to one or more of the piezoelectric actuators 924a-c, which activates the piezoelectric assembly 942 and causes piezoelectric crystals within the piezoelectric assembly 942 to at least partially deform. As the piezoelectric crystals deform, the piezoelectric assembly 942 extends (or moves) longitudinally towards the distal end 222 of the handpiece 900a. When the piezoelectric assembly 942 extends distally, a distal end of the piezoelectric assembly 942 engages with the spring 944, which presses against the anchor 946 coupled to the shaft 236. Thus, the longitudinal movement of the piezoelectric assembly 942 results in longitudinal movement of the shaft 236 and the inner cutting member 206. The cutting assembly 214 is then in the closed, first state once the spring 944 has extended the inner cutting member 206 distally.

When a voltage is no longer applied to the piezoelectric assembly 942, the deformed piezoelectric crystals return to their initial, undeformed static state. As such, the piezoelectric assembly 942 recedes (or retracts), causing movement of the spring 944 to reduce and the cutting assembly 214 to move to the open, second state shown in FIG. 3B above. Such activation and deactivation of the piezoelectric assembly 942 causes the spring 944 to achieve resonance and transition the cutting assembly 214 from the first state illustrated by FIG. 3A, to the second state illustrated by FIG. 3B above.

Thus, the cutting assembly 214 in the handpiece 900a is configured to transition (or move) between the first state and second state shown in FIGS. 3A and 3B, respectively, by terminating and applying a voltage (e.g., a sinusoidal voltage) provided to the piezoelectric assembly 942. Such actuation of the cutting assembly 214 allows the cutting assembly 214 to be driven in resonance to achieve a longitudinal oscillatory motion and necessary stroke of the cutting assembly 214.

Note that FIG. 9A only illustrates one possible arrangement where a piezoelectric assembly 942 is coupled to the shaft 236, and that many other arrangements are also within the scope of the disclosure. For example, in certain embodiments, the piezoelectric assembly 942 may be disposed towards the distal end 222, as shown by handpiece 900b in FIG. 9B. Positioning the piezoelectric assembly 942 towards the distal end 222 as shown in FIG. 9B allows the piezoelectric assembly 942 to be closer to the cutting assembly 214, thereby reducing excess vibrations.

Additionally or alternatively, the piezoelectric assembly 942 may include more than or less than three piezoelectric actuators 942a, 942b, 942c. In certain embodiments, the anchor 946 may be coupled to the shaft 236 using other coupling mechanisms such as, a pin, a screw, an adhesive, etc.

The use of the piezoelectric assembly 942 to actuate the cutting assembly 214, as described herein, enables a handpiece to achieve greater cut-rates. As a result of increased cut-rates provided by the piezoelectric assembly 942, the handpiece experiences improved efficiency when removing vitreous from the eye.

The present disclosure may be embodied in other specific forms without departing from its spirit or essential characteristics. The described embodiments are to be considered in all respects only as illustrative and not restrictive. The scope of the present disclosure is, therefore, indicated by the appended Claims rather than by this Detailed Description. All changes which come within the meaning and range of equivalency of the Claims are to be embraced within their scope.

Reference throughout this specification to features, advantages, or similar language does not imply that all the features and advantages that may be realized with the present disclosure should be or are in any single embodiment of the disclosure. Rather, language referring to the features and advantages is understood to mean that a specific feature, advantage, or characteristic described in connection with an embodiment is included in at least one embodiment of the present disclosure. Thus, discussions of the features and advantages, and similar language, throughout this specification may, but do not necessarily, refer to the same embodiment.

Furthermore, the described features, advantages, and characteristics of the disclosure may be combined in any suitable manner in one or more embodiments. One skilled in the relevant art will recognize, in light of the description herein, that the disclosure can be practiced without one or more of the specific features or advantages of a particular embodiment. In other instances, additional features and advantages may be recognized in certain embodiments that may not be present in all embodiments of the present disclosure.

Reference throughout this specification to “one embodiment”, “an embodiment”, or similar language means that a particular feature, structure, or characteristic described in connection with the indicated embodiment is included in at least one embodiment of the present disclosure. Thus, the phrases “in one embodiment”, “in an embodiment”, and similar language throughout this specification may, but do not necessarily, all refer to the same embodiment.

The foregoing description is provided to enable any person skilled in the art to practice the various embodiments described herein. Various modifications to these embodiments will be readily apparent to those skilled in the art, and the generic principles defined herein may be applied to other embodiments. Thus, the claims are not intended to be limited to the embodiments shown herein but are to be accorded the full scope consistent with the language of the claims.

What is claimed is:

1. A system comprising:
a vitrectomy handpiece comprising:
a cutting assembly; and
a diaphragm disposed in a drive chamber and coupled to the cutting assembly; and
a pneumatic assembly coupled to the vitrectomy handpiece, the pneumatic assembly comprising:
an airflow controlling member configured to direct air to the drive chamber to move the diaphragm, thereby actuating the cutting assembly; and
an actuating assembly configured to actuate the airflow controlling member.

2. The system of claim 1, wherein:
the pneumatic assembly further comprises:
a housing having a plurality of slots that define a plurality of airflow paths therethrough, and wherein the airflow controlling member is disposed within the housing between the plurality of slots; and
the actuating assembly comprises:
a first piezoelectric actuator disposed at a first end of the housing; and
a second piezoelectric actuator disposed at a second end of the housing that is opposite the first end,
wherein the first piezoelectric actuator and the second piezoelectric actuator alternately engage with opposing ends of the airflow controlling member to move the airflow controlling member, thereby alternately directing the air through an extension airflow path and a retraction airflow path.

3. The system of claim 2, wherein the pneumatic assembly further comprises:
a first spring disposed between the first piezoelectric actuator and the airflow controlling member; and
a second spring disposed between the second piezoelectric actuator and the airflow controlling member.

4. The system of claim 2, wherein the airflow controlling member has a plurality of apertures therethrough, and wherein the plurality of apertures are configured to align with the plurality of slots to allow the air to flow therethrough.

5. The system of claim 1, wherein the actuating assembly comprises a piezoelectric fan that alternately directs the air through an extension airflow path defined by a first tube and a retraction airflow path defined by a second tube.

6. The system of claim 5, wherein:
the airflow controlling member is a tube configured to output the air;
the piezoelectric fan is coupled to the tube; and
actuation of the piezoelectric fan moves the tube to alternately direct the air through the first tube and the second tube.

7. The system of claim 5, wherein the airflow controlling member is the piezoelectric fan and actuation of the piezoelectric fan moves the piezoelectric fan to alternately block the air from entering the first tube and the second tube.

8. The system of claim 1, wherein:
the airflow controlling member is a wheel having a plurality of indentations and a plurality of protrusions that are alternately disposed circumferentially around the wheel; and
the actuating assembly is a motor configured to rotate the wheel, wherein:
rotation of the wheel causes the plurality of indentations and the plurality of protrusions to alternately align with an extension airflow path and a retraction airflow path,
the plurality of indentations allow the air to flow through the extension airflow path and the retraction airflow path, and
the plurality of protrusions block the air from flowing through the extension airflow path the retraction airflow path.

9. The system of claim 1, wherein:
the pneumatic assembly further comprises:
a housing having a plurality of slots that define a plurality of airflow paths therethrough;
the airflow controlling member comprises a first protrusion and a second protrusion disposed within the housing; and
the actuating assembly comprises:
a first piezoelectric actuator coupled to the first protrusion; and
a second piezoelectric actuator coupled to the second protrusion,
wherein the first piezoelectric actuator and the second piezoelectric actuator are alternately activated to move the first protrusion and the second protrusion into an extension airflow path and a retraction airflow path.

10. The system of claim 1, wherein:
the pneumatic assembly is disposed in a surgical console; and
the vitrectomy handpiece is coupled to the pneumatic assembly via a first tube that defines an extension airflow path and a second tube that defines a retraction airflow path.

11. A vitrectomy handpiece comprising:
a cutting assembly;
a diaphragm disposed in a drive chamber and coupled to the cutting assembly; and
a pneumatic assembly coupled to the cutting assembly, the pneumatic assembly comprising:
an airflow controlling member configured to direct air to the drive chamber to move the diaphragm, thereby actuating the cutting assembly; and
an actuating assembly configured to actuate the airflow controlling member.

12. The vitrectomy handpiece of claim 11, wherein:
the pneumatic assembly further comprises:
a housing having a plurality of slots that define a plurality of airflow paths therethrough;
the airflow controlling member comprises a first protrusion and a second protrusion disposed within the housing; and
the actuating assembly comprises:
a first piezoelectric actuator coupled to the first protrusion; and
a second piezoelectric actuator coupled to the second protrusion,
wherein the first piezoelectric actuator and the second piezoelectric actuator are alternately activated to move the first protrusion and the second protrusion into an extension airflow path and a retraction airflow path.

13. The vitrectomy handpiece of claim 11, wherein the actuating assembly comprises a piezoelectric fan that alternately directs the air through an extension airflow path defined by a first tube and a retraction airflow path defined by a second tube.

14. The vitrectomy handpiece of claim 11, wherein:
the airflow controlling member is a wheel having a plurality of indentations and a plurality of protrusions that are alternately disposed circumferentially around the wheel; and
the actuating assembly is a motor configured to rotate the wheel, wherein:
the rotation of the wheel causes the plurality of indentations and the plurality of protrusions to alternately align with an extension airflow path and a retraction airflow path,
the plurality of indentations allow the air to flow through the extension airflow path and the retraction airflow path, and
the plurality of protrusions block the air from flowing through the extension airflow path the retraction airflow path.

15. A vitrectomy handpiece comprising:
a handpiece configured to be removably coupled to a cutting assembly, the handpiece comprising:
an actuating assembly coupled to the cutting assembly and configured to actuate the cutting assembly to cut vitreous of an eye, the actuating assembly comprising:
a piezoelectric assembly; and
a spring coupled to the piezoelectric assembly and the cutting assembly,
wherein the piezoelectric assembly engages with the spring to cause movement of the cutting assembly.
